(19) United States
(12) Patent Application Publication
(10) Pub. No.: US 2026/0359946 A1
(43) Pub. Date: Nov. 12, 2026

(54) PATIENT-SPECIFIC UNCERTAINTY AND BIAS QUANTIFICATION AND DECISION SUPPORT FOR DEEP NEURAL NETWORKS USED IN CLINICAL X-RAY COMPUTED TOMOGRAPHY EXAMS

(71) Applicant: Mayo Foundation for Medical Education and Research, Rochester, MN (US)

(72) Inventors: Hao Gong, Rochester, MN (US); Lifeng Yu, Byron, MN (US); Shuai Leng, Rochester, MN (US); Cynthia H. McCollough, Byron, MN (US)

(21) Appl. No.: 19/133,154

(22) PCT Filed: Nov. 27, 2023
(86) PCT No.: PCT/US2023/081200

§ 371 Date: May 27, 2025

Related U.S. Application Data
(60) Provisional application No. 63/385,028, filed on Nov. 27, 2022.

(51) Int. Cl. G06T 5/70; G06T 5/60; G06T 7/00; G16H 15/00; G16H 30/40

(52) U.S. Cl. CPC G06T 5/70; G06T 5/60; G06T 7/0012; G16H 15/00; G16H 30/40; G06T 2207/10081; G06T 2207/20081; G06T 2207/20084; G06T 2207/30096

(57) ABSTRACT

Patient-specific uncertainty (e.g., total uncertainty, knowledge uncertainty, data uncertainty) and bias in medical images that have been processed using an artificial intelligence (“AI”) model, such a neural network-based model, are quantified. A unified workflow is used to quantify the performance uncertainties and bias of neural network models, including those used for reconstructing images, denoising images, material decomposition, lesion detection, and other AI-based processing tasks. The quantification of the uncertainties and bias can then be used to facilitate human decision-making processes in routine clinical practice.

### CROSS-REFERENCE TO RELATED APPLICATIONS

This application claims the benefit of U.S. Provisional Patent Application Ser. No. 63/385,028, filed on Nov. 27, 2022, and entitled “PATIENT-SPECIFIC UNCERTAINTY AND BIAS QUANTIFICATION AND DECISION SUPPORT FOR DEEP NEURAL NETWORKS USED IN CLINICAL X-RAY COMPUTED TOMOGRAPHY EXAMS,” which is herein incorporated by reference in its entirety.

### BACKGROUND

Many computed tomography (“CT”) system manufacturers, software companies, and academic research groups have been developing deep neural network (“NN”) and other artificial intelligence (“AI”) models for various clinical CT applications, such as reducing image noise and artifacts that obscure the underlying anatomical structures or pathological features; virtually enhancing or suppressing target tissues or contrast media; and detecting target diseases. These rapidly evolving methods have shown great potential for advancing clinical x-ray CT practice. Nonetheless, the performance of these methods suffers from uncertainties due to strong non-linearity and the black-box nature of NN models (e.g., convolutional neural networks). Consequently, radiologists may make a wrong diagnosis when basing their decision on an AI-processed CT image with high uncertainty. Therefore, these uncertainties should be quantified and monitored, which could enable optimizing the development and management of these NN models used in clinical practice. Further, by measuring these uncertainty levels, the downstream decision-making process (e.g., diagnosis, or management of routine CT protocols) can be better supported.

### SUMMARY OF THE DISCLOSURE

The present disclosure addresses the aforementioned drawbacks by providing a method for quantifying neural network uncertainty (e.g., total uncertainty, knowledge uncertainty, data uncertainty) and bias from medical images processed with a neural network. The method includes accessing medical image data with a computer system, and accessing a neural network with the computer system. The neural network has been trained on training data to perform an artificial intelligence (“AI”)-based processing task on medical images. AI-processed medical image data are generated by inputting the medical image data to the neural network, generating an output as the AI-processed medical image data. Uncertainty of the neural network is quantified using a knowledge distillation framework in which a student neural network is trained using the neural network. The uncertainty of the neural network is estimated from the student neural network. Model bias of the neural network is quantified using a simulation extrapolation and the AI-processed medical image data. A report based on the quantified uncertainty, the quantified model bias of the neural network, and the AI-processed medical image data is then generated. The report indicates a confidence of using the AI-processed medical image data in a clinical decision making process.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 illustrates an example patient-specific quantification of uncertainty (e.g., total uncertainty, knowledge uncertainty, data uncertainty) and bias and decision support workflow.

FIG. 2 illustrates an example workflow for uncertainty (e.g., total uncertainty, knowledge uncertainty, data uncertainty) quantification through combined knowledge distillation and variational inference.

FIG. 3 illustrates a comparison between the disclosed knowledge uncertainty quantification and an example reference.

FIG. 4 illustrates an example comparison between a bias quantification map and an example reference.

FIG. 5 is an example dual-task convolutional neural network architecture for material quantification and material classification from dual energy CT images, where the neural network architecture has been modified to directly estimate data and model uncertainty in the material quantification and classification tasks.

FIG. 6 illustrates a streamlined process for quantifying uncertainty, detecting anomalies, and assisting clinical decision making (UNION) in deep learning-based denoising (DLD).

FIGS. 7A-7C illustrate examples of original DLD outputs and UNION-derived means and total uncertainty across 4 dose level with the same underlying 5.4 mm ground glass nodule (FIG. 7A). The arrows indicate the nodule location. Boxplots of UNION-derived signal-uncertainty-ratio (SUR) per nodule per dose level are shown in FIG. 7B. SUR values tend to degrade as dose level decreased. In FIGS. 7B and 7C, GGN refers to ground glass nodule and PSN refers to partial solid nodule. FIG. 7C shows a comparison of nodule detectability across 10 experimental conditions. With UNION, AL (area under localization receiver operating characteristic curve) was systematically improved.

FIG. 8 is a flowchart setting forth the steps of an example method for quantifying neural network uncertainty (e.g., total uncertainty, knowledge uncertainty, data uncertainty) and bias and using that information to inform clinical decision support, downstream decision making processes, and the like. Additionally or alternatively, the method may detect anomalies in the model outputs and/or support clinical decision making.

FIG. 9 is a block diagram of an example system for quantifying neural network model uncertainty and bias, and for downstream clinical decision support based on those data.

FIG. 10 is a block diagram of example components that can implement the system of FIG. 9.

### DETAILED DESCRIPTION

Described here are systems and method for quantifying patient-specific uncertainty in medical images that have been processed using an artificial intelligence (“AI”) model, such as a neural network-based model or other machine learning-based model. The disclosed systems and methods provide a unified workflow to quantify the performance uncertainties and bias of neural network models, including those used for reconstructing images, denoising images, material decomposition, lesion detection, and other AI-based processing tasks. Additionally or alternatively, quantification of the uncertainties can be used to facilitate human decision-making processes in routine clinical practice. Advantageously, the systems and methods described in the present disclosure are generalizable to different neural network models (e.g., commercial neural network models), CT systems, and diagnostic CT tasks. As another advantage, the disclosed systems and methods provide a comprehensive pipeline that characterizes intrinsic uncertainty and bias of neural network models (including black-box neural network models) and exploits this information to facilitate radiologist decision-making processes.

The systems and methods described in the present disclosure are capable of evaluating several types of uncertainty in AI-processed data: data uncertainty (e.g., from errors in the inputs, from random noise in the images, etc.), which may also be referred to as aleatoric uncertainty; model uncertainty, which may also be referred to as system or knowledge uncertainty, systemic uncertainty, systematic uncertainty, and/or epistemic uncertainty (e.g., when training and testing data differ); and bias (e.g., uncertainty of neural network weights, systematic error in the results, etc.). In some instances, the quantified uncertainty may be referred to as total uncertainty, and may include one or more types of uncertainty as noted above. Maps depicting these uncertainties are generated and output to a user (e.g., a radiologist), who can review the uncertainty map(s) along with the original image(s) in order to decide on the level of confidence in their diagnosis.

Advantageously, the disclosed systems and methods work even on non-transparent and/or proprietary algorithms, such as by creating a separate duplicate model via knowledge distillation, from which uncertainty can then be quantified. The systems and methods can also work either in the projection domain or the image domain.

The uncertainties of neural network models are mainly caused by erroneous perturbations in the inputs (e.g., noise and/or artifacts in input CT images) and by the dissimilarity between training data and testing data. Based on Bayesian theory, the probability distribution p(y|x) of neural network outputs, y, with respect to a given input, x, can be formulated as the joint probability of neural network model likelihood, p(y|x, ω), and a posterior distribution, p(ω|X,Y), as:

p(y|x,X,Y)=∫p(y|x,ω)p(ω|X,Y) (1);
where ω represents the neural network model parameters (e.g., weights) learned from training samples (i.e., X: input set, Y: label set in training). If both, p(y|x,ω) and p(ω|X,Y) are known, p(y|x) can be mathematically calculated to provide a direct measurement of the uncertainty level of neural networks (e.g., uncertainty can be gauged as standard deviation of the probability distribution around a given value of x.

In some embodiments, the model likelihood, p(y|x, ω), can be approximated as a Gaussian distribution. The posterior distribution, p(ω|X,Y), on the other hand, is generally mathematically intractable. The posterior distribution can be mathematically approximated, however. For example, mathematical approaches (e.g., variational inference) can be used to approximate posterior distribution, p(ω|X,Y), using probability distributions with known characteristics. As a non-limiting example, a variational inference technique such as include Monte Carlo Dropout, Bayesian neural network, and/or deep ensemble can be used.

Nonetheless, drawback of these methods is that the neural network architecture and corresponding training data must be completely known. Another drawback of these methods is that they require re-training the original neural network models and/or modifying the neural network architecture. In practice, the target neural network architecture and the corresponding training data are not accessible (e.g., commercial, and other proprietary neural network models), which makes it impossible to directly deploy the state-of-the-art variational inference methods. In addition, there exists bias in the outputs of neural network models. It is typically impractical to directly measure bias, due to the absence of ground truth (e.g., noise-free patient CT images).

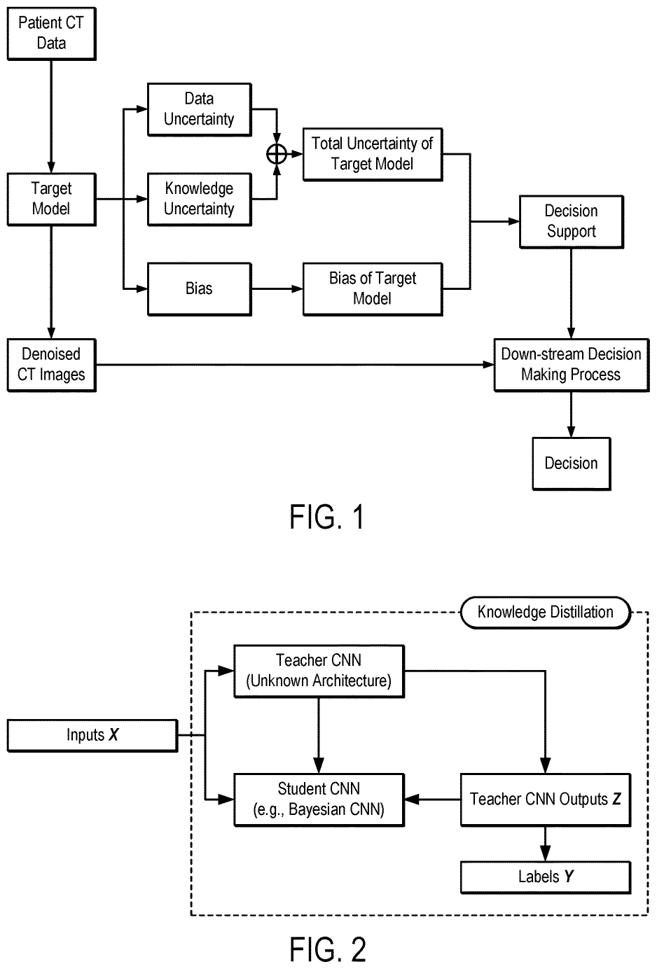

In some aspects, the systems and methods described in the present disclosure provide a unified pipeline that allows for quantifying patient-specific uncertainty (e.g., total uncertainty, knowledge uncertainty, data uncertainty) and bias while also assisting a user in the downstream decision-making process, as illustrated in FIG. 1. Thus, in some aspects, the systems and methods described in the present disclosure provide for quantifying the total uncertainty of a neural network's outputs. In some other aspects, the systems and methods described in the present disclosure provide a decision support scheme that exploits the uncertainty and bias information for radiologists' diagnostic task.

The systems and methods described in the present disclosure implement a machine learning algorithm framework using knowledge distillation and variational inference. In some embodiments, two independent sources of uncertainty can be quantified: data uncertainty and knowledge uncertainty. Moreover, using the disclosed framework, the patient CT data input to the machine learning model can be either raw projection data or image data acquired from CT scanners. Also, as noted above, the target neural network models can be models used for different CT tasks, such as image reconstruction, denoising, lesion detection, and material decomposition.

Uncertainty quantification can be performed based on a machine learning framework that integrates knowledge distillation with variational inference. In general, knowledge distillation is a procedure that transfers knowledge from a large-scale teacher neural network model to a small-scale student model, which allows the trained student model to perform similarly to the teacher model (e.g., usually in a given target task). Student models can be trained using dual labels (e.g., as shown in FIG. 2); that is, training of the student model can be based on labels from the original training set of the teacher model and based on the outputs of the teacher model. In some implementations, the student model may be a student CNN. The student CNN may include a Bayesian CNN, or the like. More generally, the student model includes a transparent AI model with a known training set and/or known architecture. When the original training set is not accessible, a new dataset with paired inputs and labels can be collected for the training of the student model. An example objective function that can be used to optimize a student models' parameters is as follows:

$$L = α \cdot {‖ {Y}_{s t u d e n t} - {Y}_{original} ‖}_{2}^{2} + ( 1 - α ) \cdot {‖ {Y}_{student} - {Y}_{teacher} ‖}_{2}^{2} ; \qquad ( 2 )$$

where α is a constant weighting factor; Y_{student} and Y_{teacher}, are the outputs of student and teacher models, respectively; and Y_{original} is the corresponding label in the original training set of the teacher model.

One of the advantages of knowledge distillation is that it can be used to realize the functionalities of some high-performance neural network models in a special device (e.g., a mobile device) that does not have sufficient computing capability to run training and/or inference of large-scale neural network models. In the systems and methods described in the present application, knowledge distillation is used to create a student model that can mimic the teacher model's behavior. The student model is then used to estimate uncertainty of the teacher model through variational inference. Because the student model is fully accessible, variational inference can be readily implemented to estimate total uncertainty.

As one non-limiting example for quantifying knowledge uncertainty, Monte Carlo Dropout can be readily integrated into the student model's architecture to quantify knowledge uncertainty. Alternatively, the student model can be implemented as Bayesian neural network (without Monte Carlo Dropout) to quantify knowledge uncertainty. Both techniques are readily applicable for target neural network models that use either patient images or raw projection data as an input.

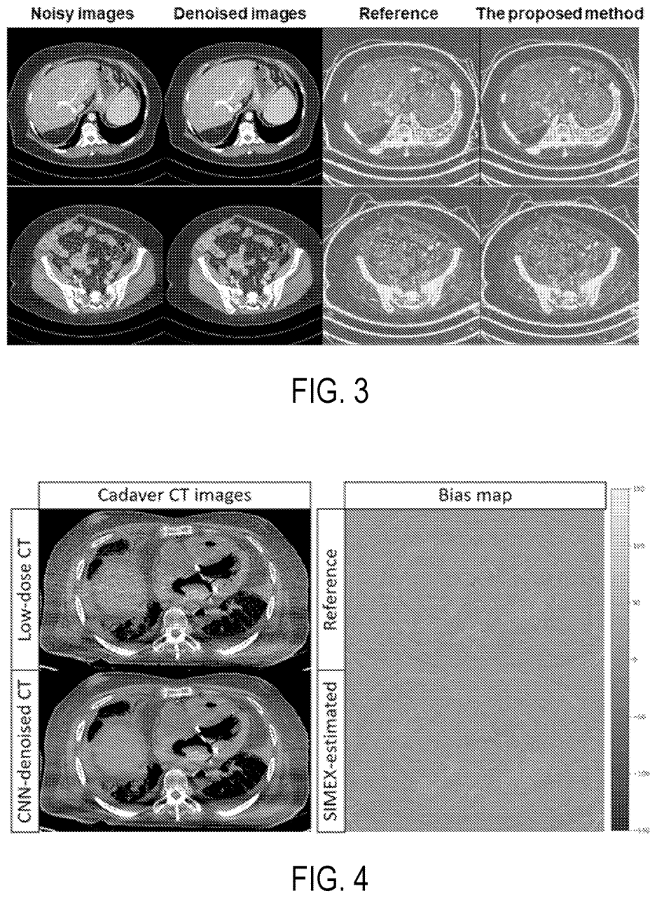

FIG. 3 illustrates a comparison between the disclosed total uncertainty (i.e., the combined data and model uncertainty) quantification and an example reference. In this example, the teacher model was a standard U-Net, and the student model was a small-scale U-Net (only quarter scale of teacher model) with bypass connection. The disclosed method was able to accurately estimate knowledge uncertainty level of the teacher model.

Based on Bayesian theorem, the probability distribution of a CNN output, y, for a given input, x, is formulated as in Eqn. (1). The posterior distribution p(ω|X,Y) is mathematically intractable, and thus may be approximated. To quantify model uncertainty, a student model (e.g., a student CNN), Φ_{S} may be created as a deep Bayesian CNN model that approximates p(ω|X,Y) through variational inference, or the like. Compared to deterministic CNNs, the Bayesian CNN applies a prior statistical distribution over each convolutional layer. Monte Carlo Dropout (i.e., applying dropout in both training and inference) can be used to implement the Bayesian CNN to effectively create a Bernoulli distribution over convolutional layers. For example, an intractable posterior distribution p(ω|X,Y) may be approximated through applying a Bernoulli distribution, q(ω), per each CNN layer (e.g., Monte Carlo dropout), as follows. A Kullback-Leibler divergence may be minimized:

L=KL(p(ω|X,Y)|q(ω) (3).

This may be equal to minimizing the negative of log evidence lower bound:

L=−∫q(ω)log p(Y|x,ω)dω+KL(q(ω)|p(ω)) (4);
following which, the approximation may be applied:

$$L = \frac{1}{N} {\sum }_{n = 1}^{N} - log p ( {y}_{n} \vert {x}_{n} , {ω}_{n} ) + η {‖ ω ‖}_{2}^{2} . \qquad ( 5 )$$

Assuming a Gaussian likelihood model and independently and identically distributed (i.i.d.) samples, for each {xₙ, yₙ, ωₙ}:

$$p ( {y}_{n} \vert {x}_{n} , {ω}_{n} ) = \frac{1}{\sqrt{2 {πσ}_{n}^{2}}} {e}^{( - \frac{{( {y}_{n} - {Φ}_{{ω}_{n}} ( {x}_{n} ) )}^{2}}{2 {σ}_{n}^{2} ( {x}_{n} )} )} . \qquad ( 6 )$$

Considering the data dependency of

$${σ}_{n}^{2} ,$$

the loss function in Eqn. (5) can be simplified as:

$$L = \frac{1}{N} {\sum }_{n = 1}^{N} ( \frac{{( {y}_{n} - {Φ}_{{ω}_{n}} ( {x}_{n} ) )}^{2}}{{σ}_{n}^{2} ( {x}_{n} )} + log {σ}^{2} ( {x}_{n} ) ) + η {‖ ω ‖}_{2}^{2} . \qquad ( 7 )$$

As a non-limiting example for quantifying data uncertainty, test-time augmentation can be used. When the target neural network models use patient images as inputs, a simplistic form of test-time augmentation can be used, which includes image rotation, flipping, mirroring, and shifting. Virtual clinical imaging trials (e.g., as described in U.S. Pat. No. 11,367,185, which is herein incorporated by reference in its entirety) can be used to provide more advanced augmentation for data uncertainty quantification. When the target neural network models use raw projection data as inputs, physics-informed test-time augmentation can be implemented, such as by varying starting angles used in CT data acquisition.

Alternatively, data uncertainty can be directly modeled. As one non-limiting example, the data uncertainty can be directly modeled by configuring the student neural network as a Bayesian neural network. For instance, the output layer of the student neural network can be a probabilistic layer that directly models the probability distribution of the neural network outputs. For instance, to estimate data uncertainty, a learnable probabilistic layer with a Gaussian prior can be used as the output layer of the student neural network, since data uncertainty is typically dependent on the inputs. In these instances, data uncertainty observed in the inputs can be directly predicted by the output layer. Advantageously, the student neural network utilized in the systems and methods described in the present disclosure can simultaneously quantify knowledge uncertainty (i.e., model uncertainty) and data uncertainty in its outputs, and no labels of “uncertainty” are needed in training.

Additionally or alternatively, a probabilistic distribution can also be applied to every convolutional layer of the student neural network. In an example implementation, the student neural network is used to mimic teacher neural network by minimizing the following loss function L_{KD}:

L_{KD}=λ·L_{hard}(Φ_{S}(X),Y)+(1−λ)·L_{soft}(Φ_{S}(X),Φ_{T}(X)) (8).

Where {X, Y} are paired inputs and labels in a training dataset (e.g., a new training set); Φ_{S}(⋅) is a student neural network; Φ_{T}(⋅) is a teacher neural network, which in some embodiments may be a non-transparent teacher neural network; L_{hard} and L_{soft} are the loss terms for hard label Y and soft label ω_{T}(X), respectively; and λ is a scalar determining the relative importance between L_{hard} and L_{soft}. When the student neural network is configured as a Bayesian neural network, the L_{hard} and L_{soft} in Eqn. (1) can be negative log likelihood. For regression tasks (e.g., image denoising, material decomposition), the negative log likelihood can be reformulated as:

$${L}_{hard} = \frac{1}{N} \sum ( \frac{\vert E [ {Φ}_{S}^{{ω}_{k}} ( {x}_{i} ) ] - {\hat{y}}_{i} \vert}{{σ}_{{y}_{i}} ( {x}_{i} ) + ϵ} + log \vert {σ}_{y i} ( {x}_{i} ) \vert ) + η \cdot {‖ {ω}_{k} ‖}_{2}^{2} ; \qquad ( 9 )$$

$${L}_{soft} = \frac{1}{N} \sum ( \frac{\vert E [ {Φ}_{S}^{{ω}_{k}} ( {x}_{i} ) ] - {Φ}_{T} ( {x}_{i} ) \vert}{{σ}_{{y}_{i}} ( {x}_{i} ) + ϵ} + log \vert {σ}_{{y}_{i}} ( {x}_{i} ) \vert ) + η \cdot {‖ {ω}_{k} ‖}_{2}^{2} ; \qquad ( 10 )$$

Where N is number of samples per mini-batch; {xᵢ, ŷᵢ} is pair of input and label for i^{th} sample;

$${Φ}_{S}^{{ω}_{k}} ( \cdot )$$

is a student neural network with randomly sampled parameter set, ωₖ; σᵢ is a predictive standard deviation (i.e., data uncertainty); η is the weight decay; and ϵ is a small scalar to prevent dividing by zero. In some implementations, a Laplace prior can be used in Eqn. (9) and (10) to maintain numerical stability in training. As another example, the loss terms of hard and soft labels can be updated as:

$${L}_{h a r d} = \frac{1}{N} {\sum }_{n = 1}^{N} ( \frac{{( {y}_{n} - {Φ}_{S , {ω}_{n}} ( {x}_{n} ) )}^{2}}{{σ}_{n}^{2} ( {x}_{n} )} + log {σ}_{n}^{2} ( {x}_{n} ) ) + η {‖ ω ‖}_{2}^{2} ; \qquad ( 11 )$$

$${L}_{soft} = \frac{1}{N} {\sum }_{n = 1}^{N} ( \frac{{( {Φ}_{T} ( {x}_{n} ) - {Φ}_{S , {ω}_{n}} ( {x}_{n} ) )}^{2}}{{σ}_{n}^{2} ( {x}_{n} )} + log {σ}_{n}^{2} ( {x}_{n} ) ) + η {‖ ω ‖}_{2}^{2} . \qquad ( 12 )$$

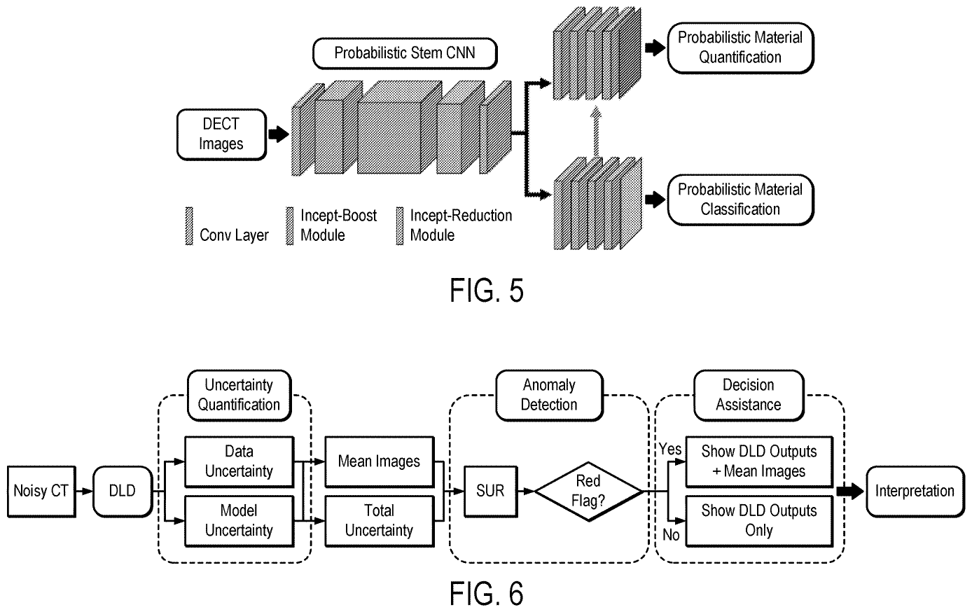

As an example, FIG. 5 illustrates a dual-task convolutional neural network that has been trained on phantom CT images to perform material quantification and material classification tasks, which has been modified to estimate data uncertainty using the techniques described above. The CNN shown in FIG. 5 uses a bifurcated architecture with two branches that concurrently conducted material classification and quantification tasks. The classification can serve as an auxiliary regularization to material quantification tasks. To quantify knowledge uncertainty, a dropout layer was paired with each convolutional layer in the CNN (dropout probability 0.1). To quantify data uncertainty in the material maps, a probabilistic output layer was used with Gaussian prior. To account for data uncertainty in the classification task, another probabilistic layer was added before Softmax layer, and the corresponding predictive mean and standard deviation was estimated through Monte Carlo sampling on logit space. In material quantification branch, the corresponding loss function applied negative log likelihood in both material maps and spectral image domain using the framework described above.

Due to approximation errors, the predictive uncertainty quantified using the systems and methods described in the present disclosure may not be well-calibrated with actual uncertainty and the expected error in some instances (e.g., Monte Carlo Dropout may underestimate model uncertainty). In these instances, a re-calibration procedure can be implemented. In general, an empirical joint probability density function of expected error and total uncertainty is created through 2D histogram binning, and bin-wise scaling factors (i.e., the ratio of the expected error and total predictive uncertainty) are derived to adjust total predictive uncertainty:

$${s}_{k} = \frac{\sqrt{\frac{1}{J} \sum {‖ E [ {y}_{j} ] - {\hat{y}}_{j} ‖}_{2}^{2}}}{\sqrt{\frac{1}{J} {σ}_{tot , j}^{2}}} ; \qquad ( 13 )$$

where sₖ is the scaling factor for the kth bin, which has J data points, and

$${σ}_{tot , j}^{2} = {σ}_{data , j}^{2} + {σ}_{model , j}^{2}$$

in the sum of variances due to data and model (i.e. knowledge) uncertainty, respectively.

To quantify model bias, the systems and methods described in the present disclosure estimate the ground truth through simulation extrapolation (“SIMEX”). SIMEX estimates the model response for noise-free input data. The outputs of a neural network model are assumed to be very close to ground truth when there is no noise presented in the input data. For example, a CT denoising neural network model is expected to preserve all image information when its inputs are noise-free. In the systems and methods described in the present disclosure, SIMEX can be implemented as follows. When reference data are not available, bias may be estimated through adapting SIMEX as:

F(λ)=φ(x′+λz′) for λ=1,2, . . . (14).

Assuming a well-trained model (e.g., a well-trained deep-learning-based denoising and reconstruction model), φ can well approximate an unbiased estimator of the ground truth, given noise-free inputs x₀: φ(x)=x₀. Varying realizations of inputs can be synthesized with increasing noise levels, x′+λz′. Thus, increased synthetic noise is added to the input data, and the corresponding neural network model outputs generated by inputting the noise-augmented input data to the neural network are collected. Then, model response for noise-free input data (denoted as “pseudo ground truth”) can be extrapolated across the neural network model outputs across different noise level. The extrapolation can be carried out using, for example different regression models (e.g., polynomial fitting and isotonic regression). The bias can then be estimated by subtracting the pseudo ground truth from the measured mean of the model outputs (i.e., an approximation of the expectation of model response) acquired from the data and knowledge uncertainty quantification. FIG. 4 illustrates an example comparison between a bias quantification map and an example reference. The disclosed method for quantifying bias demonstrates high accuracy. The mean-absolute-difference between the reference and the estimated value was <7 HU in this example.

The framework described above can be extended to provide a streamlined process that quantifies uncertainty, detects anomalies, and assists in clinical decision making. Data and model uncertainty can be quantified as described above. In anomaly detection, the ratio between the predicted mean outputs and total uncertainty, denoted as signal uncertainty ratio (SUR), can be used as a test statistic. When the mean SUR at a target image region falls below a threshold, the CNN outputs are identified as overly dispersed, and a flag can be triggered to indicate to a user that an anomaly is detected. For decision support, the predicted means can be displayed alongside original model outputs for clinical interpretation. An example workflow of this process, which quantifies uncertainty, detects anomaly, and assists clinical decision making (UNION) in deep learning-based denoising (DLD), is illustrated in FIG. 6.

In an example study, the systems and methods described in the present disclosure were tested using denoised image data generated using a deep learning-based denoising model and reader interpretations from a virtual lung nodule screening trial: 10 experimental conditions with 4 dose levels (full and simulated 10%, 25%, 50% dose), 2 nodule types (ground glass (GGN) and partial solid (PSN)), and 3 nodule sizes (3.4, 5.4, and 7.4 mm). To estimate diagnostic performance gain using the disclosed systems and methods, a validated deep-learning model observer (DLMO) was calibrated to reader performance measured on original deep learning-based denoising model outputs. The same DLMO was deployed, as a well-calibrated pseudo reader, to estimate diagnostic performance with the disclosed systems and methods. The area under the localization receiver operating characteristic curve (AL) was used as a figure of merit.

Mean images generated using the disclosed systems and methods showed more robust visualization of target nodules across different noise realizations and patients. SUR level of nodules degraded as dose level decreased (median SUR across 100% to 10% dose levels: GGN 5.53, 4.53, 3.54, 2.44, and PSN 5.5, 4.55, 3.55, 2.37). With the disclosed systems and methods, mean AL was consistently improved across all conditions (Signed Rank test p<0.05). The disclosed systems and methods yielded larger improvement at low dose and/or small nodules (e.g., AL with/without UNION at 10% dose: GGN—83.1%/75.8%, PSN—90.9%/77.8%; AL with/without UNION at 3.4 mm GGN and 50% dose: 63.2%/55.9%).

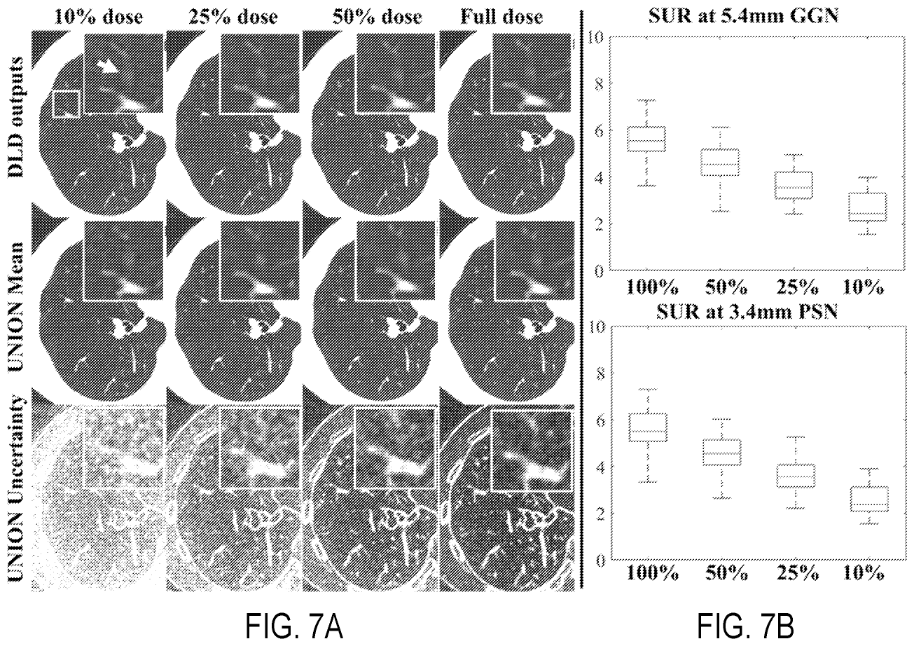

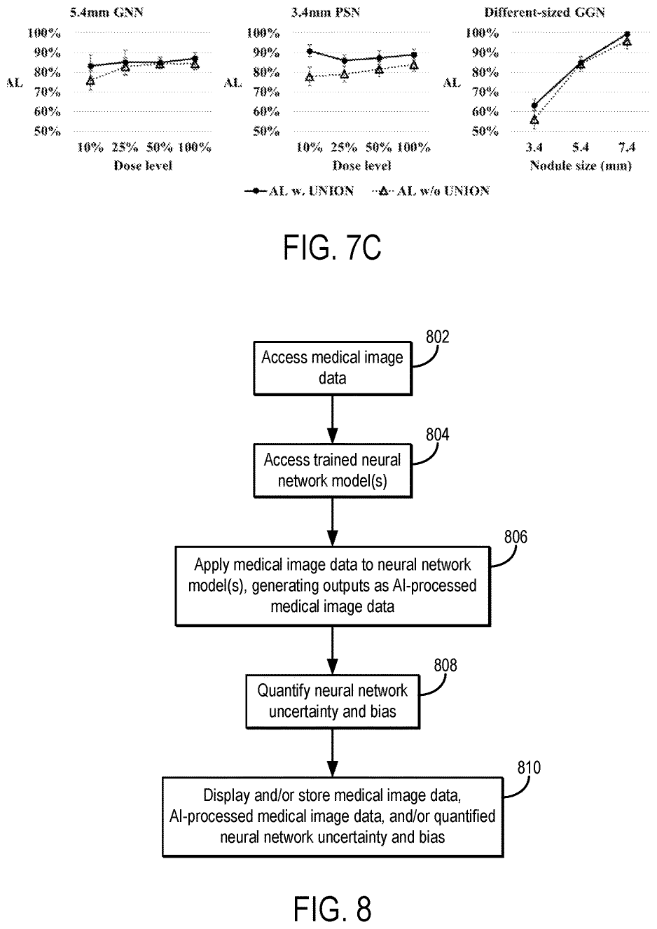

FIGS. 7A-7C illustrate examples of original DLD outputs and UNION-derived means and total uncertainty across 4 dose level with the same underlying 5.4 mm ground glass nodule (FIG. 7A). The arrows indicate the nodule location. Boxplots of UNION-derived signal-uncertainty-ratio (SUR) per nodule per dose level are shown in FIG. 7B. SUR values tend to degrade as dose level decreased. In FIGS. 7B and 7C, GGN refers to ground glass nodule and PSN refers to partial solid nodule. FIG. 7C shows a comparison of nodule detectability across 10 experimental conditions. With UNION, AL (area under localization receiver operating characteristic curve) was systematically improved.

Referring now to FIG. 8, a flowchart is illustrated as setting forth the steps of an example method for quantifying uncertainty (e.g., total uncertainty, knowledge uncertainty, data uncertainty) and bias of a neural network model and using that information to inform clinical decision support, downstream decision making processes, and the like.

The method includes accessing medical image data with a computer system, as indicated at step 802. Accessing the medical image data may include retrieving such data from a memory or other suitable data storage device or medium. Additionally or alternatively, accessing the medical image data may include acquiring such data with a medical imaging system and transferring or otherwise communicating the data to the computer system, which may be a part of the medical imaging system. The medical image data may include raw data and/or reconstructed images. For example, the medical image data may include patient CT data, which may include raw projection data acquired with a CT system and/or CT images obtained with a CT system. Additionally or alternatively, the medical image data may include medical image data (raw data, or images reconstructed from data) acquired with other medical imaging systems, such as a magnetic resonance imaging (“MRI”) system, a nuclear medicine imaging system (e.g., a single photon emission computed tomography (“SPECT”) system, a positron emission tomography (“PET”) system), an ultrasound system, or the like.

A trained neural network (or other suitable machine learning algorithm) is then accessed with the computer system, as indicated at step 804. In general, the neural network is trained, or has been trained, on training data in order to perform an AI-based processing task on medical image data. As mentioned above, the AI-based processing task may include image reconstruction, image denoising, material decomposition (in patient CT data), lesion detection, and so on.

Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs.

The medical image data are then input to the one or more trained neural networks, generating output as AI-processed medical image data, as indicated at step 806. For example, the AI-processed medical image data may include reconstructed images, denoised images, segmented images, and so on.

Uncertainty and/or bias of the neural network(s) are also quantified, as indicated at step 808. As described above, the uncertainty may include data uncertainty, knowledge uncertainty, or both. Additionally or alternatively, the uncertainty may include other types of uncertainty, including total uncertainty. Knowledge uncertainty can be quantified based on a neural network that is constructed to incorporate knowledge distillation and variational inference, as described above. In these instances, the neural network(s) accessed at step 804 will have this architecture described above in more detail. Data uncertainty can be quantified using a test-time augmentation technique, as described above in more detail. The type of test-time augmentation used can be selected based on whether the medical image data include raw data, reconstructed images, or both. Alternatively, data uncertainty can be quantified through constructing the student neural network as a Bayesian neural network and training the Bayesian neural network with negative loglikelihood-type objective functions, as described above in more detail.

Model bias can be quantified using a SIMEX technique, as described above. Synthetic noise can be added to the medical image data, generating noise-augmented medical image data. The noise-augmented medical image data are then input to the neural network(s), generating output as noise-augmented AI-processed medical image data. The model response for noise-free input data (i.e., “pseudo ground truth”) is extrapolated across the neural network model outputs (i.e., the noise-augmented AI-processed medical image data) across different noise levels. The extrapolation can be carried out through different regression models (e.g., polynomial fitting, isotonic regression, and so on). The model bias can then be estimated by subtracting the pseudo ground truth from the measured mean of the model outputs (i.e., an approximation of the expectation of model response) acquired from the data and knowledge uncertainty quantifications.

As described above, in some implementations anomalies in the model outputs may also be detected. For example, the ratio between the predicted mean outputs and total uncertainty can be computed and denoted as a signal uncertainty ratio (SUR). When the mean SUR, or other measure of SUR, at a target image region falls below a threshold, the CNN outputs are identified as overly dispersed or otherwise containing one or more anomalies. In some implementations, a flag can be set indicating the detection of one or more anomalies in the model outputs. Additionally or alternatively, an output can be generated (e.g., a visual indication can be generated and output to a user, such as a graphical element on a graphical user interface indicating that one or more anomalies were identified in the model outputs).

The measured uncertainty and bias data can then be displayed to a user, stored for later use or further processing, or both, as indicated at step 810. As described above, the quantitative estimates of uncertainty and bias can be incorporated into the downstream decision-making process. In some embodiments, the measured uncertainty and/or bias data can be used to support a clinical decision making process. As a non-limiting example, a decision support scheme can be generated for a radiologist interpretation on neural network-denoised patient images. For instance, for a given patient case, the spatial distribution of uncertainty and bias can be visualized along with patient CT images during image interpretation; the radiologists can then adjust their decision according to the estimated uncertainty/bias level that is presented alongside the images. For example, if a suspicious region-of-interest (“ROI”) yielded high uncertainty and/or bias level, radiologists will be likely to reduce their lesion detection confidence and decision. Thresholds of uncertainty and bias can also be quantitatively optimized through virtual clinical imaging trials (as described above), to determine low and high levels of uncertainty and bias. The decision support scheme can be used to improve diagnostic performance. As described above, in some examples, the measured uncertainty and bias data may be utilized to assist with clinical decision support. For instance, the predicted means can be displayed alongside original model outputs for clinical interpretation.

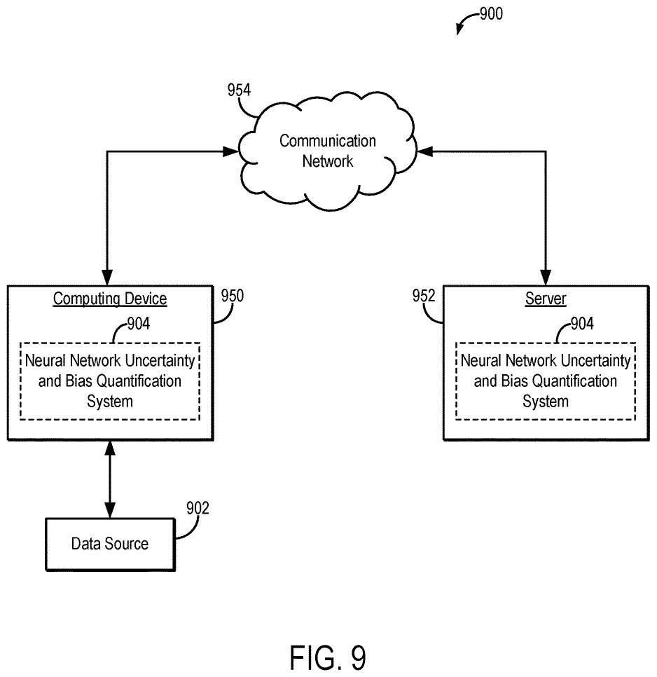

Referring now to FIG. 9, an example of a system 900 for uncertainty and bias quantification in accordance with some embodiments of the systems and methods described in the present disclosure is shown. As shown in FIG. 9, a computing device 950 can receive one or more types of data (e.g., medical image data, trained neural network parameters) from data source 902. In some embodiments, computing device 950 can execute at least a portion of a neural network uncertainty and bias quantification system 904 to quantify neural network uncertainty and bias, in addition to providing downstream clinical decision support, from data received from the data source 902.

Additionally or alternatively, in some embodiments, the computing device 950 can communicate information about data received from the data source 902 to a server 952 over a communication network 954, which can execute at least a portion of the neural network uncertainty and bias quantification system 904. In such embodiments, the server 952 can return information to the computing device 950 (and/or any other suitable computing device) indicative of an output of the neural network uncertainty and bias quantification system 904.

In some embodiments, computing device 950 and/or server 952 can be any suitable computing device or combination of devices, such as a desktop computer, a laptop computer, a smartphone, a tablet computer, a wearable computer, a server computer, a virtual machine being executed by a physical computing device, and so on. The computing device 950 and/or server 952 can also reconstruct images from the data.

In some embodiments, data source 902 can be any suitable source of data (e.g., measurement data, images reconstructed from measurement data, processed image data), such as a medical imaging system, another computing device (e.g., a server storing measurement data, images reconstructed from measurement data, processed image data), and so on. In some embodiments, data source 902 can be local to computing device 950. For example, data source 902 can be incorporated with computing device 950 (e.g., computing device 950 can be configured as part of a device for measuring, recording, estimating, acquiring, or otherwise collecting or storing data). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 954 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

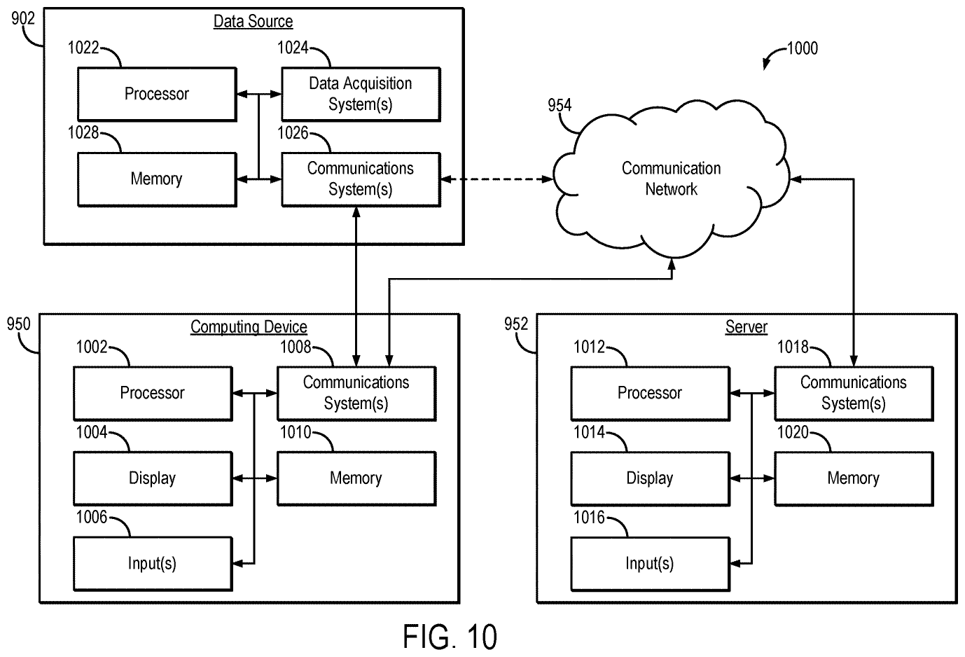

Referring now to FIG. 10, an example of hardware 1000 that can be used to implement data source 902, computing device 950, and server 952 in accordance with some embodiments of the systems and methods described in the present disclosure is shown.

As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (“CPU”), a graphics processing unit (“GPU”), and so on. In some embodiments, display 1004 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware, and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on. For example, the processor 1002 and the memory 1010 can be configured to perform the methods described herein (e.g., the workflow of FIG. 1, the workflow of FIG. 2, the workflow of FIG. 6, the method of FIG. 8).

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as an LCD screen, LED display, OLED display, electrophoretic display, a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware, and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, the server 952 is configured to perform the methods described in the present disclosure. For example, the processor 1012 and memory 1020 can be configured to perform the methods described herein (e.g., the workflow of FIG. 1, the workflow of FIG. 2, the workflow of FIG. 6, the method of FIG. 8).

In some embodiments, data source 902 can include a processor 1022, one or more data acquisition systems 1024, one or more communications systems 1026, and/or memory 1028. In some embodiments, processor 1022 can be any suitable hardware processor or combination of processors, such as a CPU, a GPU, and so on. In some embodiments, the one or more data acquisition systems 1024 are generally configured to acquire data, images, or both, and can include a CT system, and MRI system, or the like. Additionally or alternatively, in some embodiments, the one or more data acquisition systems 1024 can include any suitable hardware, firmware, and/or software for coupling to and/or controlling operations of a medical imaging system (e.g., a CT system, an MRI system). In some embodiments, one or more portions of the data acquisition system(s) 1024 can be removable and/or replaceable.

Note that, although not shown, data source 902 can include any suitable inputs and/or outputs. For example, data source 902 can include input devices and/or sensors that can be used to receive user input, such as a keyboard, a mouse, a touchscreen, a microphone, a trackpad, a trackball, and so on. As another example, data source 902 can include any suitable display devices, such as an LCD screen, an LED display, an OLED display, an electrophoretic display, a computer monitor, a touchscreen, a television, etc., one or more speakers, and so on.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more data acquisition systems 1024, and/or receive data from the one or more data acquisition systems 1024; to generate images from data; present content (e.g., data, images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1028 can have encoded thereon, or otherwise stored therein, a program for controlling operation of data source 902. In such embodiments, processor 1022 can execute at least a portion of the program to generate images, transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for quantifying neural network uncertainty and bias from medical images processed with a neural network, the method comprising:
(a) accessing medical image data with a computer system;
(b) accessing a neural network with the computer system, wherein the neural network has been trained on training data to perform an artificial intelligence (AI)-based processing task on medical images,
(c) generating AI-processed medical image data by inputting the medical image data to the neural network, generating an output as the AI-processed medical image data;
(d) quantifying uncertainty of the neural network using a knowledge distillation framework in which a student neural network is trained using the neural network, wherein the uncertainty of the neural network is estimated from the student neural network;
(e) quantifying model bias of the neural network using a simulation extrapolation and the AI-processed medical image data;
(f) generating a report based on the quantified uncertainty, the quantified model bias of the neural network, and the AI-processed medical image data, wherein the report indicates a confidence of using the AI-processed medical image data in a clinical decision making process.

2. The method of claim 1, wherein the uncertainty of the neural network is estimated from the student neural network using variational inference.

3. The method of claim 1, wherein the uncertainty comprises one of knowledge uncertainty or data uncertainty.

4. The method of claim 3, wherein the uncertainty comprises knowledge uncertainty and the knowledge uncertainty is estimated based on a Monte Carlo dropout incorporated into the student neural network model architecture.

5. The method of claim 3, wherein the student neural network is a Bayesian neural network.

6. (canceled)

7. The method of claim 3, wherein the uncertainty comprises data uncertainty and the data uncertainty is quantified using a test-time augmentation.

8. The method of claim 7, wherein the medical image data comprise medical images and the test-time augmentation comprises generating augmented medical images.

9. The method of claim 8, wherein the augmented medical images are generated based on at least one of image rotations, image flipping, image mirroring, or image shifting.

10. The method of claim 7, wherein the medical image data comprise raw projection data acquired with a computed tomography (CT) system and the test-time augmentation comprises generating augmented raw projection data by varying starting angles of a CT data acquisition.

11. The method of claim 3, wherein the uncertainty comprises data uncertainty and the data uncertainty is quantified by directly modeling the data uncertainty through the student neural network.

12. The method of claim 11, wherein the student neural network includes an output layer that is a probabilistic layer that directly models the data uncertainty as a probability distribution of the student neural network outputs.

13. The method of claim 12, wherein the student neural network comprises a Bayesian neural network.

14. The method of claim 11, wherein the student neural network is a convolutional neural network comprises at least one convolutional layer, wherein a known probability distribution is applied to each of the at least one convolutional layers to improve the quantification of the data uncertainty.

15. The method of claim 1, wherein the quantifying the model bias using simulation extrapolation comprises:
generating noise-augmented medical image data by adding synthetic noise to the medical image data;
generating noise-augmented output data by inputting the noise-augmented medical image data to the neural network;
extrapolating the AI-processed medical image data across the noise-augmented output data for different noise levels; and
quantifying the model bias by subtracting the AI-processed medical image data from a mean of the noise-augmented output data.

16. The method of claim 1, wherein generating the report comprises displaying the quantified uncertainty, the quantified model bias, and the AI-processed medical image data to a user via the computer system.

17. The method of claim 16, wherein the quantified uncertainty comprises a uncertainty map and the quantified model bias comprises a model bias map.

18. The method of claim 1, wherein the AI-based processing task includes one of image reconstruction, image denoising, material decomposition, image segmentation, or lesion detection.

19. The method of claim 1, wherein the medical image data are acquired with one of a computed tomography (CT) system or a magnetic resonance imaging (MRI) system.

20. (canceled)

21. The method of claim 1, further comprising detecting anomalies in the AI-processed medical image data based on a signal uncertainty ratio computed as a ratio between predicted mean outputs of the neural network and total uncertainty in the quantified uncertainty.

22. A method for quantifying model data uncertainty for a neural network, the method comprising:
(a) accessing medical image data with a computer system;
(b) accessing a neural network with the computer system, wherein the neural network has been trained on training data to perform an artificial intelligence (AI)-based processing task on medical images,
(c) generating AI-processed medical image data by inputting the medical image data to the neural network, generating an output as the AI-processed medical image data;
(d) generating model uncertainty data by quantifying model uncertainty of the neural network using a knowledge distillation framework in which a student neural network is trained using the neural network, wherein the uncertainty of the neural network is estimated from the student neural network using variational inference; and
(e) generating a report based on the mode uncertainty data, wherein the report indicates a confidence of using the AI-processed medical image data in a clinical decision making process.

23. The method of claim 1, wherein the medical image data comprise multi-energy computed tomography (CT) data acquired with a CT system and the AI-based processing task includes material decomposition.

24. The method of claim 1, wherein the student neural network jointly penalizes data uncertainty and bias.

25. The method of claim 24, wherein the bias is penalized using a loss function that computes a difference between an expected mean of outputs of the AI-based processing task and ground truth labels.

26. The method of claim 25, wherein the data uncertainty is penalized by modeling the data uncertainty using an output layer of the student neural network that is a probabilistic layer that directly models the data uncertainty as a probability distribution of the student neural network outputs.
